(19) United States
(12) Patent Application Publication
(10) Pub. No.: US 2019/0231203 A1
(43) Pub. Date: Aug. 1, 2019

(54) HEAD WEARABLE UNIT HAVING A CONNECTOR TO A NEURAL INTERFACE

(71) Applicant: Fraunhofer-Gesellschaft zur Foerderung der angewandten Forschung e.V., Munich (DE)

(72) Inventor: Tamás HARCZOS, Ilmenau (DE)

(21) Appl. No.: 16/375,813

(22) Filed: Apr. 4, 2019

Related U.S. Application Data
(63) Continuation of application No. PCT/EP2017/075555, filed on Oct. 6, 2017.

(30) Foreign Application Priority Data
Oct. 7, 2016 (DE) 102016219511.5

(51) Int. Cl. A61B 5/04; A61N 1/375; A61B 5/00

(52) U.S. Cl. CPC A61B 5/04001; A61N 1/375; A61B 5/6831; A61B 5/6814; A61B 5/6803

(57) ABSTRACT

A head wearable unit has a shape of headphone or headband. The head wearable unit has a connector to a neural interface, a processor for processing signals received from/to be transmitted via the neural interface as well as a power supply.

### CROSS-REFERENCE TO RELATED APPLICATIONS

This application is a continuation of copending International Application No. PCT/EP2017/075555, filed Oct. 6, 2017, which is incorporated herein by reference in its entirety, and additionally claims priority from German Application No. 102016219511.5, filed Oct. 7, 2016, which is also incorporated herein by reference in its entirety.

### BACKGROUND OF THE INVENTION

Embodiments of the present invention refer to a head wearable unit having the shape of a headphone or headband and comprising a connector for connecting a neural interface.

A neural interface is typically an implanted unit staying in close contact to a specific nerve, or nerve fibers, or neurons of an implantee. The neural interface may act as a stimulator (e.g. prosthetic sensory device) or a neural recording device, or can be a combination of both.

Cochlear implant, retinal implant, brainstem implant, a cortical implant, an interface for deep brain stimulation and/or an interface for a Vagus nerve stimulation are examples of neural stimulators. A neural recording device may record signals from the cortex or the peripheral nervous system. Additionally, there are none-implanted neural interfaces like a scalp unit, also referred to as electroencephalography (EEG) unit, configured to record the neural activity from externally.

Thanks to ever-advancing miniaturization of computational technology and good available biocompatibility, neural prostheses and in particular sensory prostheses like cochlear and retinal implants are booming. Though these implants principally work, they could perform considerably better if they would make use of computational models already available for neural processing of various modalities [1][2][3]. However, the real-time simulation of advanced models of neural processing would consume much more energy [4]. Unfortunately, there is no Moore's law for batteries [5], which means that energy sources cannot be made smaller at the scale needed to build—the currently typical—behind-the-ear implant processors in their usual size, which would use advanced models. Therefore, there is a need for an improved approach.

An objective of the present invention is to provide a concept to enable avoiding the bottle-neck of the limited battery capacity and the limited processor performance.

### SUMMARY

According to an embodiment, a head wearable unit having the shape of a headphone or headband may have: at least one connector for connecting at least one neural interface; one or more processing units for processing signals to be transmitted towards the at least one neural interface or for processing signals received from the at least one neural interface; and a power supply.

According to another embodiment, a head wearable unit having the shape of a headphone or headband may have: at least one scalp unit as neural interface configured to receive the neural activity; one or more processing units for processing signals received from the at least one neural interface; and a power supply.

Embodiments of the present invention provide the head wearable unit having the shape of a headphone or headband. The head wearable unit comprises at least one connector, like an electromagnetic radiation connector or, in general, a wireless or wired connector for connecting a neural interface, like a cochlear implant. The unit further comprises a (single- or multi-core) processor for processing the signals to be received from the neural interface or to be transmitted via the neural interface. Additionally, the unit comprises a power supply, such as a battery, e.g. a rechargeable battery, or a kind of energy harvester such as a solar panel. Here, the connection may have the purpose to communicate with the neural interface and/or to provide electrical energy to same.

Embodiments of the present invention are based on the principle that the “headphone processor” helps to increase the computational power supported by the higher capacity battery easily embedded in the form factor of a headphone or headband. Several advanced signal processing schemes for cochlear implants (Cis) have been developed, the obstacle for practical body-worn implantation having been processing power and battery life. These obstacles can be overcome when integrating the power supply and the processor into a headphone or headband. From the design point of view, this shape also makes sense, since headphones have become lifestyle products, so that it is trendy to wear and be seen wearing headphones in public. This headphone/headband has additional advantages, which will be discussed with respect to further embodiments.

The connection between the connector (part of the headphone) and the implant (typically arranged some millimeters under the cranium or the scalp) may, according to embodiments, be realized using electromagnetic radiation (e.g. induction or radio frequency link), i.e. wirelessly, or using electricity, i.e. wired, or using light either via a translucent or an optical fiber based transmission (or any combination of these). A percutaneous connection enables such a wired or fiber based transmission, while a transcutaneous connection enables a wired transmission.

Another embodiment provides a head wearable unit comprising one or more surround sensors. According to embodiments, the surround sensors may comprise one microphone, a plurality of microphones, at least two microphones pointing to different directions and/or at least three microphones configured to perform beamforming. Here, the advantage of the headphone shape is that the microphones can be spaced further apart and may also be directed in different directions such that the special sound field may be reproduced better. Here, the shape has a second advantage, namely that the one unit can provide signals to the user for both ears. It should be noted, though, that it is not necessary to connect the cochlear nerves of both sides (left and right) via neural interfaces. For example, one ear may be coupled using the above described connector and a CI as a neural interface, wherein the other ear may be coupled to a common acoustic interface of the headphone. Here, according to further embodiments, the processor of the headphone may be configured to perform a binaural processing of audio signals received via one or more microphones in order to output the binaural synthesis via the connectors and neural interfaces. This enables, advantageously, the provision of spatial information of the acoustic signal to the user.

According to another embodiment, the surround sensor may comprise one or more visual sensors, such as cameras. In this case, the neural interface is typically not a CI but a retina implant. Thus, the processor processes the signals from the cameras and outputs same via the connector to the neural interface/retina implant. Here, the headphone design also makes sense, since this enables a good positioning for the visual sensor.

According to further embodiments, the power supply may comprise a battery or rechargeable battery, wherein the recharging may, according to additional embodiments, be performed using an internal device, namely an energy harvester such as a solar panel. According to an embodiment, the percutaneous or transcutaneous connection is used only for supplying the implant or implants with energy, e.g. for charging their internal batteries, should they be fully implanted. In this case, the processor takes on the task of the charging electronics, wherein the signal to be transmitted to the neural interface is an energy signal.

Another embodiment provides a head wearable unit comprising a communication interface, such as a wireless interface, enabling transmission of signals from the outside via the connector and the neural interface to the user. Vice-versa, the neural interface may be, as discussed above, a neural recording device. In the latter environment, the communication interface enables transmission of signals received from the neural interface to an external device, e.g. a medical device. This environment allows substitution of the commonly used head sensor comprising neural interfaces by a more comfortable element. Note that in this case the neural interface may be realized as scalp unit, i.e. not as an implanted part.

According to an embodiment, the scalp unit or the neural recording device enables in combination with a stimulator like a CI to implement a feedback loop. This has the advantage that the physiological reaction of the user to the stimulation can be directly measured, e.g. to adapt the stimulation procedure.

An embodiment of the present invention provides the head wearable unit having the shape of a headphone or headband. The head wearable unit comprises a scalp unit as neural interface configured to receive the neural activity, a processor for processing the signals to be received from the neural interface and a power supply.

### BRIEF DESCRIPTION OF THE DRAWINGS

Embodiments of the present invention will subsequently be discussed referring to the enclosed drawings in which:

FIG. 1a shows a schematic representation of a head wearable unit having the shape of a headphone for connecting a neural stimulator as neural interface according to a first embodiment;

FIG. 1b shows a schematic representation of a head wearable unit having the shape of a headband for connecting a neural stimulator as neural interface according to a second embodiment;

FIG. 1c shows a schematic representation of a head wearable unit having the shape of a headphone for connecting a neural recording device as neural interface according to a third embodiment;

FIG. 2 shows a head wearable unit having the shape of a headphone according to an enhanced embodiment; and

FIG. 3 shows a (part of a) head wearable unit having the shape of a headphone according to another enhanced embodiment.

### DETAILED DESCRIPTION OF THE INVENTION

Embodiments of the present invention will be discussed in detail referring to the Figures. Here, the same references numerals are provided to the same or similar objects so that the description thereof is mutually applicable and interchangeable.

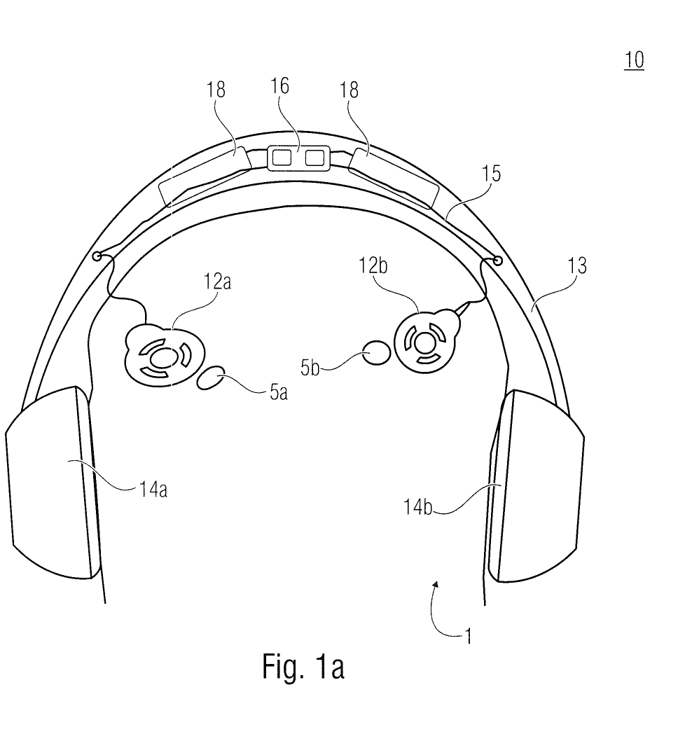

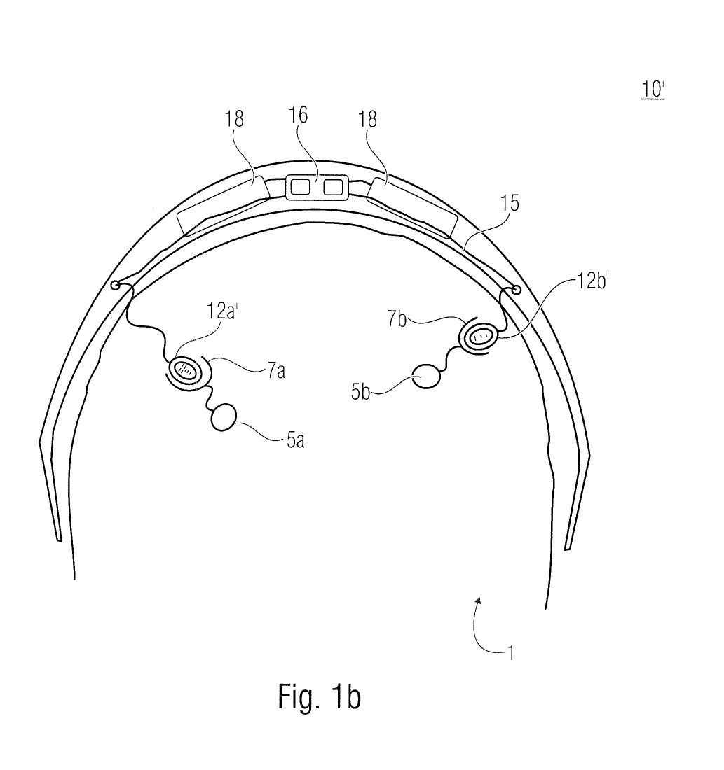

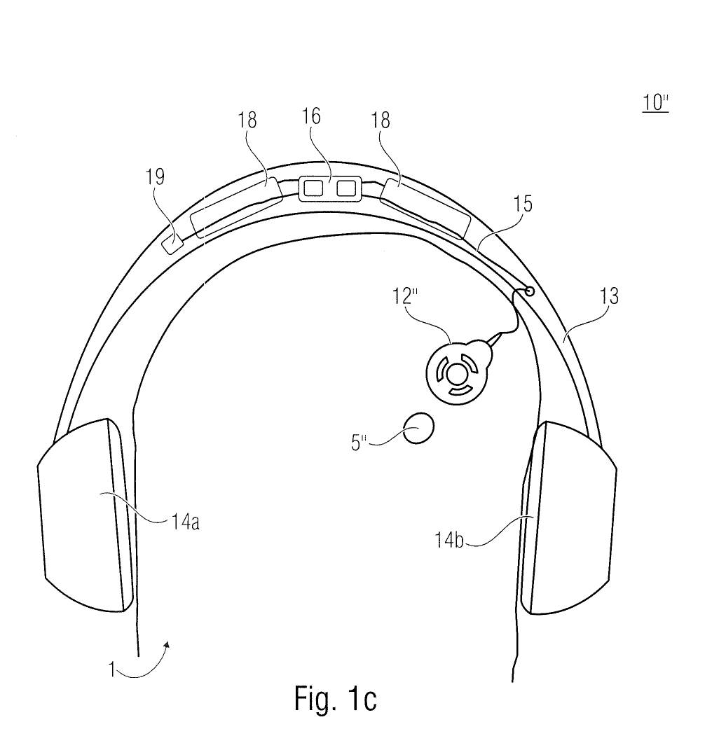

FIG. 1a shows a headphone 10 having two earphones 14a and 14b. In contrast to a conventional headphone, this headphone 10 comprises one or more connectors 12a and 12b for neural interfaces 5a and 5b, like Cls. Additionally, the headphone 10 comprises a processor 16 and a power supply 18.

The connectors 12a and 12b, here transcutaneous communication connectors, are configured to communicate with cochlear implants 5a and 5b. The two cochlear implants 5a and 5b, one for the right ear and one for the left ear, are implanted into the head of the user 1 and in close connection to the cochlear nerves, also referred to as auditory or acoustic nerves, so as to act as neural stimulators. Due to the typical positioning of cochlear implants, the connectors 12a and 12b are arranged in the proximity of the ears of the user 1.

The connectors 12a and 12b are configured to communicate with cochlear implants 5a and 5b. Thus, the connectors 12a and 12b may comprise a coil, an antenna or an inductive interface (in general: near field communication interface) configured to communicate from the outside of the head of the user 1 with the cochlear implant 5a and 5b arranged within the head. Here, each implant 5a and 5b may optionally have a counterpart (not shown) for the connector 12a and 12b. The counterpart may be arranged within the implant 5a and 5b or as separate entity arranged under the skin and connected to the implant 5a and 5b, e.g. using thin wires. Note that the connectors 12a and 12b may—alternatively or additionally—be configured to supply the implants 5a and 5b with energy. According to an alternative embodiment, an optical connection may be established.

The processor 16, which processes the signals for the connectors 12a and 12b (cf. wiring 15), e.g. an A31S ARM Cortex-A7 CPU, is arranged within the headgear 13 of the headphone 10. Here, also the power supply 18, e.g. a rechargeable battery pack, is arranged. The headgear 13 or in general, the headphone 10, provides enough space for high computational power processor along with batteries with high capacity. Below, the benefits of the neural prostheses having a new form factor will be discussed:

In [2] an auditory model-based CI signal processing strategy, which was shown to deliver significant benefits for CI users [4] including better pitch discrimination and increased speech and music quality (as perceived by the CI users) has been introduced.

Simulations also pointed out that binaural use of the novel strategy would allow CI users 1 to perform much better sound source localization tasks. The computational bottleneck of the algorithm was shown to be the auditory model. Though an average PC would be able to simulate the model in real-time, a contemporary CI processor 16 would not. Recently, the effects of exchanging the currently employed basilar membrane sub-model with a simpler one (cf. [4], pp. 123-124) have been studied. It was found that, qualitatively, the resulting stimulation patterns did not differ remarkably from the original ones (however, quantification is still to be done). Furthermore, the resulting compound model could be simulated about 80% faster than before (cf. [4], pp. 80-81). This increase would still not allow for direct use in current CI processors 16, but tests (calculating stimulation patterns for 20 CI electrodes) showed that the resulting algorithm runs smoothly on an A31S ARM Cortex-A7 CPU, a mid-range, low power, 18×18×0.65 mm processor, primarily used in tablets and smart phones. When it comes to substantially more stimulation channels than in current CI systems, for example, in future optical cochlear implants [6], current processors will inevitably be the bottleneck in computing bio-inspired stimulation patterns. It seems that a cheap CPU 16 like the one above could open whole new worlds of signal processing possibilities for neural prostheses.

The electrical power consumed could easily be provided by lithium-ion batteries as power supply 18 built into the headgear 13 or loudspeaker housing 14a/14b.

Another motivation factor for using a headphone as carrier for connectors 12a and 12b to connect the CIs/neural interfaces 5a and 5b is that the headphones have the potential to become a lifestyle product. While various headphones already have this status, visible parts (like the behind-the-ear, or BTE, processor) of cochlear implant systems are in the process of transition from invisible to eye-catching. Makers of CIs now offer their processors in various colors, while also providing colorful covers [7] and (via 3rd party) highly customizable skins [8]. These facts hint that a combination of the two, a CI processor 16 in a headphone enclosure 10, could indeed be well accepted by CI users.

Additional motivation factors are the anatomical issues. Though current CI processors are customizable in appearance, in size and shape they are not. Unfortunately, a non-neglectable number of CI users 1 have problems wearing their BTE processor(s), because of incompatibilities between processor shape and anatomical properties of their pinna(e). Currently, the only available workaround for this problem is to fall back on a body-worn processor, which means more cables and, typically, degraded quality of audio input, since the microphone(s) of the body-worn unit do not necessarily point towards the targeted sound source. A headphone enclosure 10 with customizable ear pad cushions 14ac and 14bc could become a genuine alternative.

Note that the entities integrated into the headphone 10, like the one or more neural connectors 12a and 12b, the processor 16 and the power supply 18 are connected to each other by wires 15, as illustrated within FIG. 1.

With respect to FIG. 1b, another head wearable unit, namely a headband 10′ will be discussed. FIG. 1b shows a headband 10′ comparable to the headphone 10 of FIG. 1a, but having a different shape.

The headband 10′ comprises percutaneous connectors 12a′ and 12b′ instead of the transcutaneous connectors 12a and 12b. Percutaneous connectors 12a′ and 12b′ have a counterpart 7a and 7b arranged under the scalp of the user 1, wherein the counterparts 7a and 7b are connected to the Cls 5a and 5b by wires. The other entities 15, 16 and 18 of the headband 10′ are the same as discussed with respect to the embodiment of FIG. 1a.

Of course, transcutaneous connectors 12a and 12b can also be used in combination with a headband 10′ or, vice versa, percutaneous connectors 12a′ and 12b′ can be used together with the headphone 10. Even a combination of a transcutaneous and a percutaneous connector within one device would be possible.

FIG. 1c shows a head wearable unit 10″ comparable to the head wearable unit 10; however, it is connected to a different neural interface 5″ using the connector 12″. Here, a neural interface 5″ is used instead of the neural interfaces 5a and 5b, where 5′″ is a neural recording device, recording neural activity from e.g. the cortex. The recording device (e.g. an array of electrodes) is in contact with or coupled to the cortex and configured to receive and monitor neural activity, especially related to the cortex. Thus, here the neural interface 5″ is not necessarily provided next to the ears, but may be provided somewhere else on the head of the user 1.

In this embodiment, the unit 10″ has the purpose to receive and monitor brain activities. The received brain activities can be processed using the processor 16 or stored using a memory included with the processor 16.

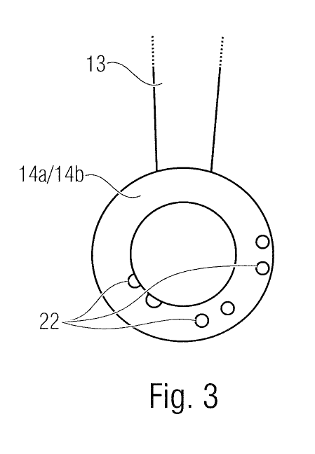

As shown e.g. in [12] it would also be easily conceivable to incorporate retro- or periauricular recording electrodes 22 into the ear pad cushions 14a/14b for electroencephalography (EEG) purposes, as illustrated by FIG. 3. These could support objective and/or closed-loop methods of fitting the prosthetic device's parameters.

In further embodiments, the head wearable unit 10″ comprises an interface 19, e.g. a computer interface which may be realized as wireless or wired. The interface 19 enables the output of the data recorded by the processor using the neural interface 5″ and the (transcutaneous or percutaneous) connector 12″. The interface 19 can further be used to connect the wearable unit 10″ and via same the Cls 5a and 5b with an audio source, e.g. Bluetooth audio. Another use case for the interface 19 is the updating or editing of the software used by the processor. Here, new features can be implemented by the software update, afterwards. Additionally, a fitting procedure can be performed, wherein fitting typically comprises the amending of the parameters used for the processing.

The increased size of the housing 13 (in comparison to a BTE enclosure) would also allow for better wireless connectivity 19: not only could an increased number of (and more sophisticated) antennas fit into the device, but also transmitters and receivers entailing direct line of sight (like infrared) could be placed easily. Furthermore, safety limits for radio transmission power could be complied with more easily by placing antennas further away from the skull.

Although, in above embodiments, the neural interface 5a, 5b, 5″ has been discussed in context of a cochlear implant and in context of a cortical neural recording device, it is apparent, that also other interfaces such as visual implants, auditory implants, cognitive implants or in general, brain-computer interfaces may be used. Here, it does not make a difference whether the neural interface enables the transmission of data from the external to the user 1 or, vice-versa from the user 1 to external.

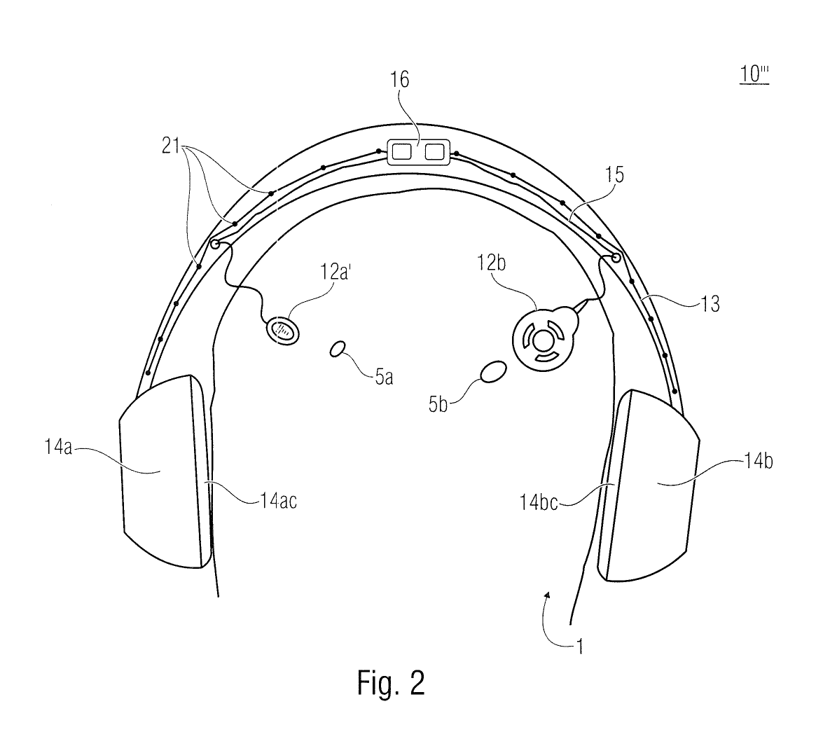

With respect to FIG. 2, an enhanced embodiment of the headphone 10 will be discussed. FIG. 2 shows the headphone 10′″, which substantially corresponds to the headphone 10, wherein the headphone 10′″ comprises an additional surround sensor 21.

The surround sensor 21 may be a microphone or advantageously a microphone arrangement enabling reception of an acoustic signal from the surrounding, to process the same using the processor 16 and to transmit the processed signals to the user 1 by the one or more cochlear implants 5a and 5b via the connectors 12a′ and 12b.

Here, the headphone design enables more space for the integration of better microphones or more microphones into the headgear 13.

By loosening the space restriction, microphones 21 taking up slightly more space, but having better response characteristics could be used. Microphone placement during design phase could make use of the surfaces facing various directions, supporting more advanced beamforming [12] and noise suppression algorithms [13].

Since the head wearable unit is arranged so that both ears can be supplied with acoustic data, e.g. via a common transducer or via the connectors 12a/12b and the neural interfaces 5a/5b, this shape leads to additional advantages with respect to the direct interaural/interlateral connection.

In the proposed processor 16 embodiment, a direct (wired) connection between the left and right side can be realized without difficulties, which means that more sophisticated binaural processing algorithms can be implemented much easier and in a more energy-efficient way. Emulation of the medial olivocochlear reflex could be implemented easily, which could support better lateralization and improve signal-to-noise ratio in the presence of noise [10]. Binaurally coherent jitter would further improve interaural time-difference sensitivity of cochlear implantees at high pulse rates [11] thus supporting sound source localization. The interaural communication used for the above (and other binaural) algorithms would not require wireless body area networking and other wireless solutions constantly radiating the human body and taking up energy.

Additionally, it should be noted that many hearing impaired patients are fitted bimodally, which means, their left and right ears are equipped with devices based on completely different stimulation paradigm (like hearing aids, bone anchored hearing aids, direct cochlea stimulators, cochlear implants, brainstem implants). The processing units of various hearing devices could be housed and interfaced more easily in the presented enclosure.

As discussed above, according to further embodiments, the power supply (18) may be enhanced by a solar panel, which fits onto the top of the headband (13) and has the purpose to counter-act battery depletion. Alternatively, other energy harvesters, such as harvesters converting vibrations into electrical energy, may be used.

Also in the above embodiments, the head wearable unit has been discussed in context of a headphone. It should be noted the wearable unit may have a different shape, such as a headband, which enables the same advantages as the headphone.

Below, an additional embodiment, having a different configuration will be discussed. An embodiment provides a head worn enclosure resembling a headphone or headband for accommodating the processing unit of neural interfaces, enabling long-term, comfortable wearing of the device, while providing enough space for demanding electronic and other components.

According to further embodiments, the neural interface may act as a stimulator (e.g. prosthetic sensory device) or a neural recording device, or can be a combination of both. The stimulating neural interface may be connected to a neural prosthesis and may include (but is not limited to) cochlear, retinal, brainstem, and cortical implants, and devices for deep brain or Vagus nerve stimulation.

Alternatively or additionally, the neural recording interface may record from any accessible part of the brain (e.g. cortex) or peripheral nervous system (e.g. auditory nerve in the cochlea) or from the scalp (e.g. recording electrodes incorporated into the ear cushion of the head-worn enclosure resembling a headphone), where the recorded neural activity may be used for diagnostics, for brain interfaces, or for any other purpose. If the neural interface includes an auditory or visual prosthesis, then it may consist of one (unilateral) or two (bilateral) units for any sensory modality.

According to a further embodiment, the head-worn enclosure may also house sensory aids with a stimulation paradigm other than direct neural stimulation, like traditional hearing aids, acousto-optical hearing aids, bone anchored hearing aids, or direct cochlea stimulators (transferring mechanical vibration to the cochlea), in any combination with the aforementioned neural interfaces.

If the neural interface includes bilateral sensory prostheses of the same modality (e.g. bilateral cochlear implants), then the proposed enclosure provides an efficient way for sophisticated synchronized bilateral processing (in case of auditory prostheses: binaural processing), through simple cabling between the left and right end of the head-worn enclosure resembling a headphone or headband. This would render wireless body area networking and other wireless solutions constantly radiating the human body and taking up energy unnecessarily.

In the case that the neural interface includes at least one cochlear implant, due to the increased volume available for electronics and battery in the proposed enclosure, more sophisticated signal processing strategies could be applied, which are shown to provide benefits for the implantees [Harczos, 2015].

In the case of a bilateral cochlear implant housed in the proposed enclosure, emulation of the medial olivocochlear reflex could be implemented easily, which could support better lateralization and improve signal-to-noise ratio in the presence of noise [Lopez-Poveda et al, 2016]. Furthermore, binaurally coherent jitter would further improve interaural time-difference sensitivity of cochlear implantees at high pulse rates [Laback and Majdak, 2008] thus supporting sound source localization.

The increased size of the proposed enclosure for accommodating the processing unit of neural interfaces (as compared with e.g. behind-the-ear cochlear implant processor housings) would allow for placing more acoustic or visual sensors (like more microphones for advanced audio signal processing like beamforming or multi-microphone noise reduction algorithms [Kokkinakis et al., 2012], or to fit solar panels onto the top of the headphone/headband to counteract battery depletion.

While this invention has been described in terms of several embodiments, there are alterations, permutations, and equivalents which will be apparent to others skilled in the art and which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

### REFERENCES

[1] W. Nogueira, A. Katai, T. Harczos, F. Klefenz, A. Buechner, and 8. Edler, “An Auditory Model Based Strategy for Cochlear Implants,” in Proc. 29th Annu. Int. Conf IEEE EMBS, Lyon, France, 2007, pp. 4127-4130.
[2] T. Harczos, A Chilian, and P. Husar, “Making use of auditory models for better mimicking of normal hearing processes with cochlear implants: the SAM coding strategy,” IEEE Trans. Biomed. Circuits Syst, vol. 7 (4), pp. 414-425, 2013.
[3] B. S. Wilson and M. F. Dorman, “Cochlear implants: Current designs and future possibilities,” J. Rehahil. Res. Dev, vol. 45 (5), pp. 695-730, 2008.
[4] T. Harczos. “Cochlear implant electrode stimulation strategy based on a human auditory model,” PhD dissertation, Department of Electrical Engineering and Infonnation Technology, Ilmenau University of Technology, 2015.
[5] F. Schlachter, “No Moore's Law for batteries,” PNAS, vol. 110 (14), p. 5273, 2013.
[6] M Jeschke and T. Moser, “Considering optogenetic stimulation for cochlear implants,” Hear. Res, vol. 332, pp. 224-234, 2015.
[7] Online resource: http://www.cochlear.com/wps/wcm/connect/us/recipients/nucleus-6/nucleus-6-accessories/customize (last browsed on 2016 Feb. 19).
[8] Online resource: http://www.medel.com/us/skins/ (last browsed on 2016 Feb. 19).
[9] Online resource: http://www.wpzses.com/images/large/Jv/Monster-Beats-Studio-Ferrari-7 04 LRG.jpg (last browsed on 2016 Feb. 19).
[10] E. A Lopez-Poveda, A. Eustaquio-Martln, J. S. Stohl, R D. Wolford, R. Schatzer, B. S. Wilson, “A Binaural Cochlear Implant Sound Coding Strategy Inspired by the Contralateral Medial Olivocochlear Reflex,” Ear Hear, vol. 37 (3), pp. e138-e148, 2016.
[11] B. Laback and P. Majdak, “Binaural jitter improves interaural timedifierence sensitivity of cochlear implantees at high pulse rates,” PNAS, vol. 105 (2), pp. 814-817, 2008.
[12] A. Buechner, K-H. Dyballa, P. Hehrmann, S. Fredelake, and T. Lenarz, “Advanced Beamforrner.; for Cochlear Implant Users: Acute Measurement of Speech Perception in Challenging Listening Conditions,” PLoS ONE, vol. 9 (4), 9 pages, DOI: 10.1371/journal.pone.0095542, 2014.
[13] K. Kokkinakis, B. Azimi, Y. Hu, and D. R Friedland, “Single and Multiple Microphone Noise Reduction Strategies in Cochlear Implants,” Trends in Amplification, vol. 16 (2), 102-116, 2012.
[14] S. Debener, R. Emkes, M. De Vos, M. Bleichner, “Unobtrusive ambulatory EEG using a smartphone and flexible printed.

1. A head wearable unit comprising the shape of a headphone or headband, comprising:
at least two connectors for connecting at least two neural interfaces and configured to communicate with the at least two neural interfaces;
one processing unit for processing signals to be transmitted towards the at least two neural interface; and
a power supply.

2. The head wearable unit according to claim 1, wherein at least one connector is realized as transcutaneous connector or percutaneous connector.

3. The head wearable unit according to claim 1, wherein the at least one connector is an electromagnetic radiation connector configured to wirelessly communicate with neural interface or to wirelessly provide electrical energy to the neural interface.

4. The head wearable unit according to claim 1, wherein the at least one connector is configured to use a radio signal, an electromagnetic signal, inductive signal or a light signal for establishing the connection to the connecting neural interface.

5. The head wearable unit according to claim 1, wherein the neural interface comprises a neural stimulator.

6. The head wearable unit according to claim 1, wherein the neural interface comprises a stimulating device or an implant placed onto, into, or in proximity of a nerve, a auditory nerve, optic nerve, brainstem, cortex, Vagus nerve, or deep brain.

7. The head wearable unit according to claim 1, wherein the neural interface comprises a neural recording device or an implant placed onto, into, or in proximity of a specific nerve or brain area and configured to record neural activity.

8. The head wearable unit according to claim 1, wherein the neural interface comprises a neural recording device recording neural activity from the cortex or peripheral nervous system.

9. The head wearable unit according to claim 1, further comprising a scalp unit configured to receive the neural activity.

10. The head wearable unit according to claim 1, wherein the head wearable unit comprises one or more surround sensors.

11. The head wearable unit according to claim 10, wherein the one or more surround sensors comprise one microphone, a plurality of microphones, at least two microphones pointing to different directions or at least three microphones configured for perform beamforming.

12. The head wearable unit according to claim 10, wherein the processing unit is configured to perform a binaural processing of audio signals received via the one or more microphones in order to output the binaural synthesis via the neural interface coupled to the head wearable unit using the connectors.

13. The head wearable unit according to claim 7, wherein the surround sensor comprises a at least one visual sensor, and wherein the processing unit is configured to forward a visual signal received via the visual sensor to the neural interface coupled to the head wearable unit using the connectors.

14. The head wearable unit according to claim 1, wherein the head wearable unit comprises a communication unit in order to communicate to peripheral devices or in order to receive a signal via the communication interface and to forward the signal via the neural interface coupled to the head wearable unit using the connectors or to forward a signal from the neural interface coupled to the head wearable unit using the connectors via the communication interface.

15. The head wearable unit according to claim 1, wherein the power supply comprises a battery or a rechargeable battery.

16. The head wearable unit according to claim 1, wherein the power supply comprises an energy harvester or a solar unit.

17. The head wearable unit according to claim 10, wherein the one or more surround sensors comprise at least two microphones pointing to different directions or at least three microphones configured for performing beamforming.

18. (canceled)

19. A head wearable unit comprising the shape of a headphone or headband, comprising:
at least two connectors for connecting at least two cochlear implants as neural interfaces and configured to communicate with the at least two cochlear implants;
one or more microphones;
one processing unit for processing signals to be transmitted towards the at least two neural interface and configured to perform a binaural processing of audio signals received via the one or more microphones in order to output the binaural synthesis using the at least two connectors; and
a power supply.
